(19) United States
(12) Patent Application Publication
(10) Pub. No.: US 2024/0389921 A1
(43) Pub. Date: Nov. 28, 2024

(54) DEVICE AND SYSTEM FOR DETECTING HEART RHYTHM ABNORMALITIES

(71) Applicant: National University of Ireland, Galway, Galway (IE)

(72) Inventors: Oisín MCGRATH, Galway (IE); Eddie MCDAID, Galway (IE); Patrick CONWAY, Galway (IE); Mark BRUZZI, Galway (IE); Gurkan DOGAN, Galway (IE)

(21) Appl. No.: 18/694,498

(22) PCT Filed: Mar. 29, 2022
(86) PCT No.: PCT/EP2022/058343

§ 371 Date: Mar. 22, 2024

(30) Foreign Application Priority Data
Sep. 22, 2021 (WO) PCT/EP2021/076129

(51) Int. Cl. A61B 5/346; A61B 5/00; A61B 5/024; A61B 5/256; A61B 5/28

(52) U.S. Cl. CPC A61B 5/346; A61B 5/02416; A61B 5/256; A61B 5/28; A61B 5/6824; A61B 5/6831; A61B 5/7221; A61B 5/7264; A61B 5/7292; A61B 2560/0443; A61B 2560/0468

(57) ABSTRACT

A wearable device for detecting heart rhythm abnormalities, comprising: at least one pulse oximeter configured to measure optically a bloodstream so as to monitor peak-to-peak (P-P) pulse timings; one ECG sensor having two or more pairs of dry electrodes and configured to measure two or more voltage signals in relation to heart rhythm activity, each voltage signal being measured across one electrode pair; and a processor unit; wherein in response to an irregularity in P-P pulse timings, the processor unit is operable to: activate the ECG sensor by polling, periodically and sequentially, each of the two or more electrode pairs; determine an electrode pair for data recording; and record voltage signal data measured through the determined electrode pair.

### FIELD OF THE INVENTION

The present invention relates to methods and systems for long-term monitoring of heart rhythm and detection of heart rhythm abnormalities, in particular for reliable and accurate detection of intermittent/asymptomatic atrial fibrillation.

### BACKGROUND TO THE INVENTION

Atrial fibrillation (AFib) is the most common heart rhythm irregularity, affecting over 33.5 million individuals globally, and is the source of significant preventable annual healthcare costs worldwide. The pathology causes a decrease in the efficiency of the heart's ability to pump blood, potentially resulting in clot formation. AFib is responsible for 50% of all fatal ischemic strokes, and sufferers are at a 5 times higher risk of stroke. Silent AFib constitutes up to 60% of all AFib and greatly extends the time to diagnosis due to the intermittent and asymptomatic nature of the AFib events. Early detection of this form of AFib presents challenges in the form of continuous recording duration, which are hampered by monitoring device convenience, type, environment of use, and wearer-compliance.

The current gold standard approach for monitoring and recording heart waveforms non-invasively is electrocardiogramacords the electrical activity of the heart and stores the waveform, which is typically printed out to a screen or document. Conventional wearable ECG monitors are not an ideal solution for monitoring periods which span more than a small number of days, and they are generally unable to record the heart waveform reliably during excessive motion, or while wet. In addition, ECG monitors tend to be large, inconvenient, and power hungry, or require wearer interaction to function.

Pulse oximetry is an alternative approach to measuring heart activity, but its utility has thus far been limited to measuring pulse rate and blood oxygenation levels rather than full heart rhythm. Pulse oximetry operates by sampling the bloodstream with light pulses and measuring property changes (e.g., intensity) of light pulses passing through or reflecting from the bloodstream. For example, by measuring intensity changes of light with varying wavelengths, the haemoglobin/deoxyhaemoglobin gradient change representing blood volume in a vessel at any given point in time can be monitored/measured. This data can provide in-depth information on cardiovascular functionality with the application of advanced processing methods.

Pulse oximeter sensors are featured in many sports wearables such as sports trackers or smartwatches today. These platforms typically offer a host of other features such as GPS, fitness applications, Bluetooth, Wi-Fi, screen display and more in order to compete within the consumer market. The pulse oximeter sensors incorporated into these devices are low performance, and are incapable of providing the data that cardiologists need in order to detect waveform abnormalities which may be indicative of AFib. For example, existing pulse oximetry-based devices typically have a low sampling/polling frequency in the range of 5 Hz to 215 Hz. A poor temporal resolution, resulting from a low sampling frequency, makes such devices unable to resolve certain temporal features of a heart rhythm waveform that are important for determining AFib. As such, the output waveform readings from such devices are of lower quality and lower reliability. In addition to this, the necessity of hosting a wide range of functionality to maintain a competitive edge within the consumer market limits battery life, curtailing a key feature necessary to reliably monitor for infrequently occurring heart rhythm abnormalities over long durations.

Due to specific difficulties in identifying silent AFib, patients can wait years to obtain a diagnosis using current wearable heart rhythm monitoring methods such as Holter monitor and patch ECGs, leaving sufferers at significant risk of stroke, and leaving health systems exposed to significant cost increases. The intermittent nature of silent AFib means that it is difficult to detect accurately and reliably without a system which can monitor continuously in everyday settings for time periods extending into weeks and months.

### SUMMARY OF THE INVENTION

Hence, it is the objective of this disclosure to provide a method and a system that are based on a combination of pulse oximetry and ECG, and are dedicated to long-term continuous heart rhythm monitoring. The proposed method and system are capable of reliably detecting heart rhythm abnormalities e.g., heart arrhythmia while obviating or mitigating most or all of the aforementioned problems associated with existing monitoring devices.

In accordance with a first aspect of the present invention, there is provided a wearable device for detecting heart rhythm abnormalities, comprising: at least one pulse oximeter configured to measure optically a bloodstream so as to monitor peak-to-peak (P-P) pulse timings; one ECG sensor having two or more pairs of dry electrodes and configured to measure two or more voltage signals in relation to heart rhythm activity, each voltage signal being measured across one electrode pair; and a processor unit; wherein in response to an irregularity in P-P pulse timings, the processor unit is operable to: activate the ECG sensor by polling, periodically and sequentially, each of the two or more electrode pairs; determine an electrode pair for data recording; and record voltage signal data measured through the determined electrode pair.

In accordance with a second aspect of the present invention, there is provided a system for detecting heart rhythm abnormalities, comprising a wearable device as claimed in any of claims 1 to 13; an online analysis platform configured to process and analyse data transferred from the wearable device in order to determine heart rhythm abnormalities.

In accordance with a third aspect of the present invention, there is provided a method for improving ECG data quality, comprising: detecting an irregularity in P-P pulse timings by at least one pulse oximeters; activating an ECG sensor having two or more pairs of dry electrodes; polling, periodically and sequentially, each of the two or more electrode pairs; calculating a signal-to-noise ratio (SNR) for each electrode pair based on voltage signal data measured through the corresponding electrode pair in a period of polling time; identifying one or more electrode pairs that have a SNR equal to or higher than a SNR threshold; selecting the electrode pair having the highest SNR from the identified one or more electrode pairs; and recording voltage signal data measured through the determined electrode pair.

### DETAILED DESCRIPTION

Embodiments of the present invention will now be described by way of example only and with reference to the accompanying drawings, in which:

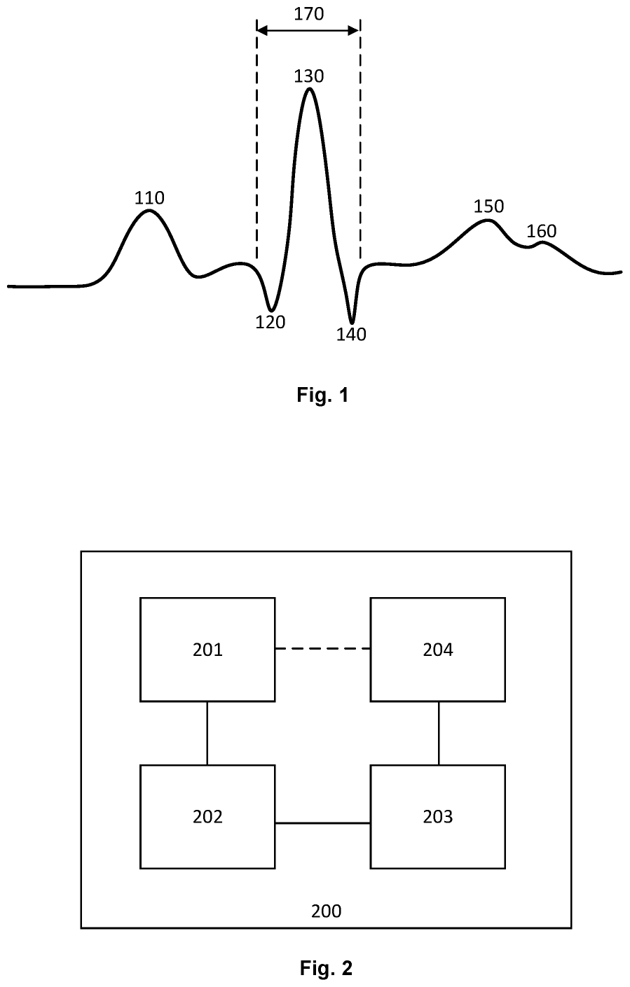

FIG. 1 depicts features of an example heart rhythm waveform;

FIG. 2 depicts a block diagram of a system for detecting heart rhythm abnormalities in accordance with an embodiment;

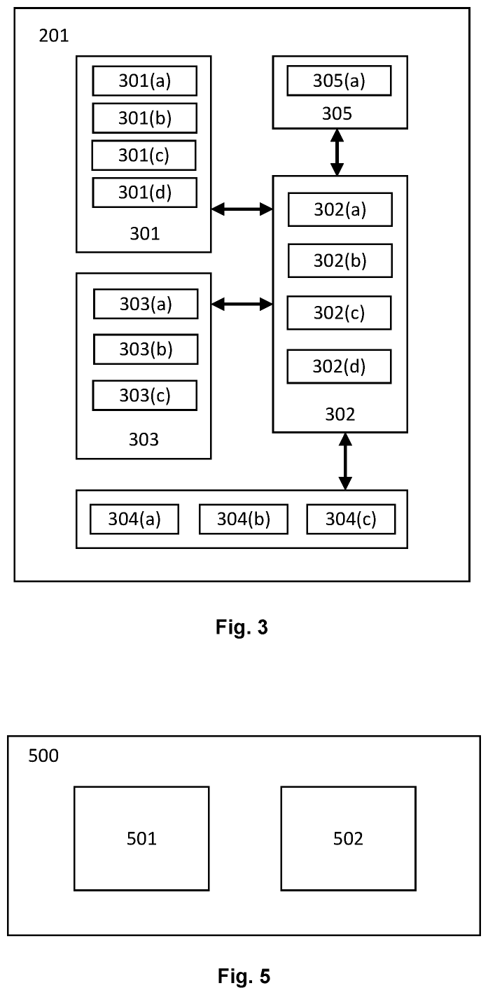

FIG. 3 depicts a block diagram of the sensing device used in the system for detecting heart rhythm abnormalities in accordance with an embodiment;

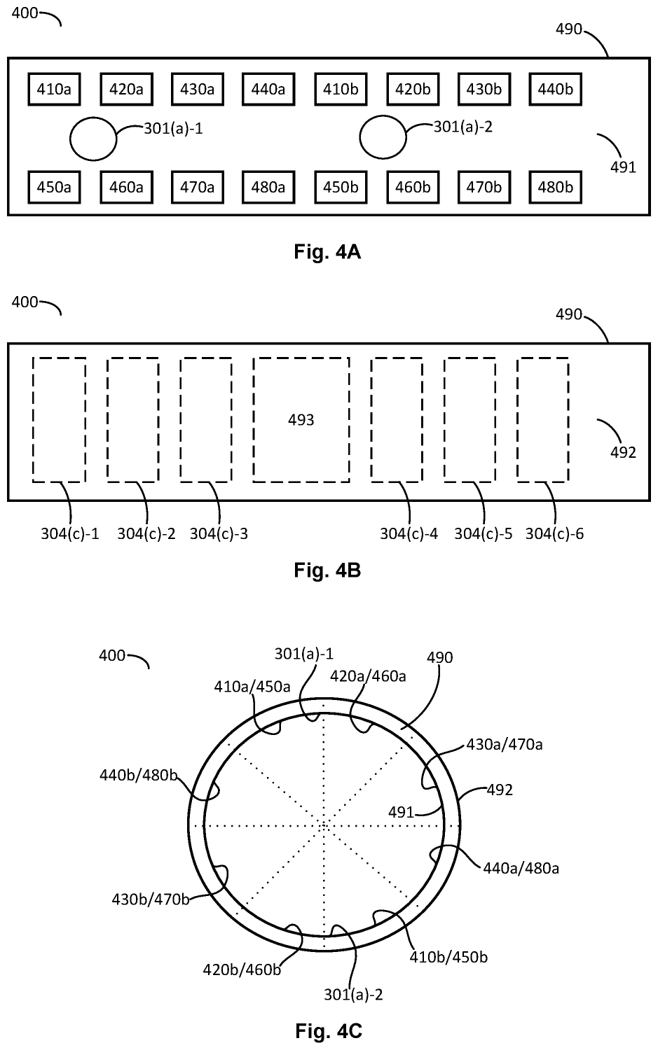

FIG. 4A depicts a plan view of the inner side of a detachable armband in an unfolded state, the armband comprising two pulse oximeters and one ECG sensor with multiple (e.g., eight) pairs of dry electrodes in accordance with an embodiment;

FIG. 4B depicts a plan view of the outer side of the detachable armband (e.g., as shown in FIG. 10a) in an unfolded state in accordance with an embodiment;

FIG. 4C depicts a side view of the detachable armband (e.g., as shown in FIG. 10a) in a folded state in accordance with an embodiment;

FIG. 5 depicts a block diagram of the charging device used in the system for detecting heart rhythm abnormalities in accordance with an embodiment;

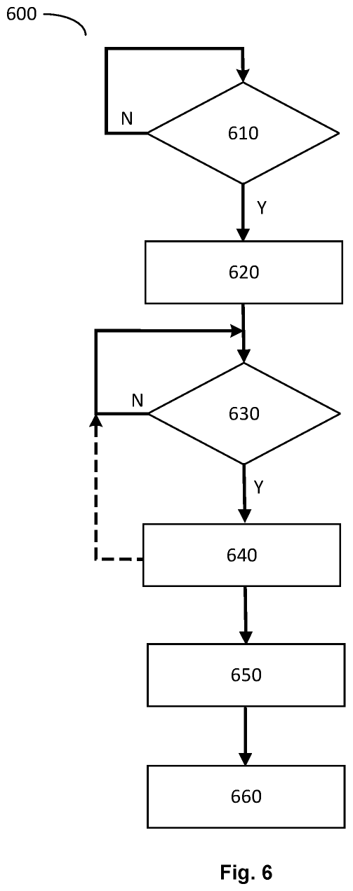

FIG. 6 depicts a flowchart of a self-starting and auto-reporting algorithm employed by the sensing device comprising a pulse oximeter and an ECG sensor in accordance with an embodiment;

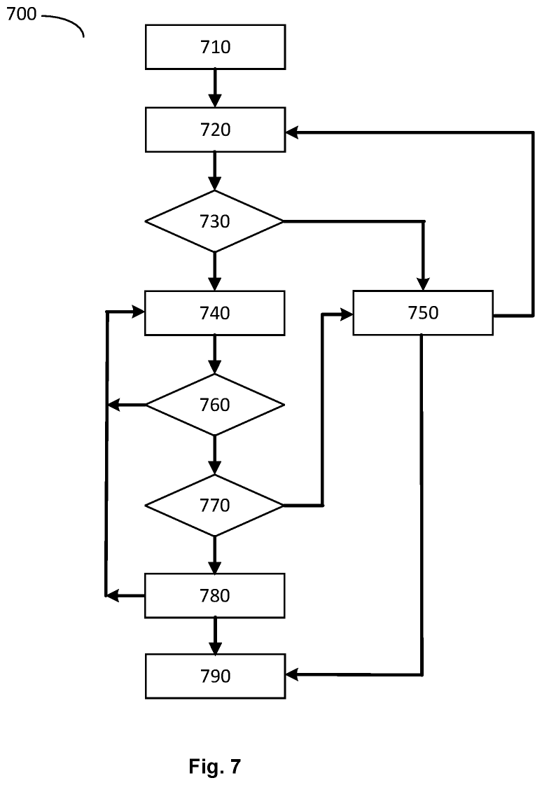

FIG. 7 depicts a flowchart of dynamic-switching algorithm employed by the sensing device comprising a pulse oximeter and an ECG sensor in accordance with an embodiment;

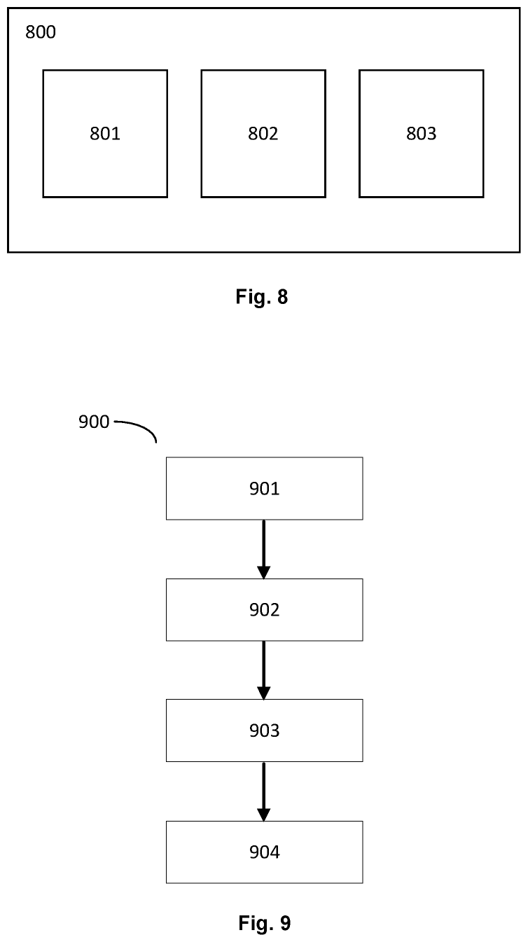

FIG. 8 depicts a block diagram of the online analysis platform in accordance with an embodiment; and

FIG. 9 depicts a flowchart of deterministic analysis performed by the data analysis function block of the online analysis platform in accordance with an embodiment.

With reference to FIG. 1, there is illustrated an example electrocardiogram of a single heartbeat. The electrocardiogram is a tracing of the electrical activity taking place within a heart. Under normal circumstances, an electrical impulse travels from the sinoatrial node, across the atrium and to the atrioventricular node and through the ventricular septum of the heart. Excited by the electrical impulse, the four chambers of the heart contract and relax in a coordinated manner. As illustrated in FIG. 1, the example electrocardiogram of a single heartbeat comprises multiple peaks and troughs representing different stages of heart beating. The peaks and troughs are denoted from the left to right as P-wave 110, Q-wave 120, R-wave 130, S-wave 140, T-wave 150 and U-wave 160, among which the P-wave 110 and the R-wave 130 are the most important features for detecting heart arrhythmia.

The P-wave 110 represents the atrial contraction and indicates the atrial depolarization. The Q-wave 120, R-wave 130 and S-wave 140 are named as QRS complex 170 which represents the electrical impulse as it spreads through the ventricles and indicates ventricular depolarization. The QRS complex 170 starts just before ventricular contraction. The T-wave 150 following the QRS complex 170 indicates ventricular repolarization. Heartbeat regularity is established by measuring time intervals between any two successive R-wave 130 peaks. Fibrillation of the atria is indicated by the absence of the P-wave 110. Measurement of irregular contractions of the atria and monitoring heart rate relative to the activity level of a patient are both important for identification of AFib. Note that not all pulsatile features indicative of some smaller waveform features displayed in FIG. 1 are easily measurable using pulse oximetry. For example, pulsatile characteristics representing the action driving the P-wave 110, Q-wave 120, S-wave 140, T-wave 150, and U-wave 160 present detection challenges due to either being caused by actions within the heart which occur before the opening of the tricuspid valve, or not resulting in large heart contractions which are detectable via haemoglobin level differences in the bloodstream.

Typically, a silent AFib patient will not physically feel any symptoms, and will not know they are at a significant risk of stroke. Hence, identification of AFib relies on a system which can be used over a prolonged period of time during everyday activities. As conventional wearable ECG is not suitable for everyday, continuous use, the invention seeks to provide everyday and a prolonged monitoring approach which can accurately and reliably capture critical AFib-indicative features, e.g., P-waves and R-waves. As mentioned above, the sampling frequency of existing pulse oximetry based devices typically lies in the range of 5 Hz and 215 Hz. A low sampling rate (and thus low temporal resolution) will only allow pulse oximeters to measure a parameter called the ‘first derivative photoplethysmogram’, which gathers information on blood oxygenation levels by measuring blood flow through large vessels. By contrast, detecting volume changes in small vessels (capillaries) using a high sampling rate (and thus high temporal resolution) will allow pulse oximeters to measure a parameter called the ‘second derivative photoplethysmogram’ (SDPPG), which can provide insights into the functionality of the heart chambers. In order to meet the demand of high temporal resolution required for high fidelity heart rhythm waveform and for accurate triggering of an ECG sensor, a pulse oximetry based monitoring device that is capable of providing a sampling rate of over 215 Hz, and preferably over 500 Hz, is highly desired.

With reference to FIG. 2, there is illustrated a block diagram of the heart arrhythmia detecting system 200 in accordance with an embodiment. The block diagram shows the core components of the system and the relationship between them. In some embodiments, the system may comprise a wearable heart rhythm monitoring device (or sensing device) 201, a charging device 202, a computing device 203, and an online analysis platform 204. In some embodiments, the charging device 202 may incorporate all the functions of the computing device 203 and thus a separate computing device will not be needed. In other embodiments, the heart arrhythmia detecting system 200 may comprise one or more other functional components that are not illustrated in the block diagram of FIG. 2. The sensing device 201 may be configured to collect data from the bloodstream of a person, e.g., a patient, which can be used later to derive a high fidelity heart rhythm waveform. The charging device 202 in connection with the computing device 203 may be configured to charge the sensing device 201 and enable data transfer from the sensing device 201 to the computing device 203. The computing device 203 in connection with the internet, may be configured to receive the data from the sensing device 201, and subsequently or simultaneously upload the data to the online analysis platform 204. In some embodiments, the online analysis platform 204 may be run on a remote server and may be configured to further process the data, analyse the processed data and finally generate an evaluation report.

Each of the above-mentioned core components will be described in detail below. With reference to FIG. 3, there is illustrated a block diagram of the functional components of the sensing device 201 and their interactions in accordance with an embodiment. In the embodiment, the sensing device 201 comprises a sensor unit 301, a processor unit 302, a memory unit 303, a power unit 304, and an interface unit 305.

In an embodiment, the sensor unit 301 may comprise at least one multi-channel pulse oximeter 301(a). The multi-channel pulse oximeter 301(a) may comprise a light emitter emitting at least two optical wavelengths. In an embodiment, the multi-channel pulse oximeter 301(a) may emit wavelengths in blue, green, yellow, red, and infrared wavelengths between 400 and 970 nm, cycling between them periodically depending on activity levels, and as charge levels vary. In an embodiment, the multi-channel pulse oximeter 301(a) may only emit two wavelengths, e.g., 660 nm and 940 nm, sequentially and periodically. The sampling frequency of the multi-channel pulse oximeter 301(a) may be controlled by the processor unit 302. The multi-channel pulse oximeter 301(a) may be operable at a high sampling rate. In an embodiment, the sampling rate may be preferable to be greater than 215 Hz. In an embodiment, the sampling rate may be preferable to be greater than 500 Hz. In an embodiment, the sampling rate may be adjustable for example, between 5 Hz and 1000 Hz, between 215 Hz and 1000, between 215 Hz and 800 Hz, or between 215 Hz and 500 Hz.

In an embodiment, the sensor unit 301 may further comprise an ECG sensor 301(b). The ECG sensor 301(b) may comprise an ECG circuit and one or more pairs of electrodes, each being electrically connected to the ECG circuit. The ECG sensor 301(b) may be configured to measure an electrical potential difference or electrical voltage between each pair of electrodes which are attached to two locations of the body surface. The electrodes may preferably be dry electrodes that can be attached directly to the body surface without requiring use of electrolytic gel. Each electrode may be in the form of a conductive pad or strip. In an embodiment, the ECG sensor 301(b) may comprise multiple pairs (i.e. two or more pairs) of dry electrodes. Multiple electrode pairs may be preferable over a single pair because more electrodes help improve the chances that one will record a good quality (e.g., high signal to noise ratio) ECG signal.

In an embodiment, the ECG sensor 301(b) may be configured such that it can be switched between two operating modes, i.e. an idling mode and an active mode. When in the idling mode, the ECG sensor 301(b) may not measure electrical voltages from multiple pairs of electrodes and thus generate no electrical voltage signals. Whereas, when in the active mode, the ECG sensor 301(b) may continuously measure the electrical voltage between one or more pairs of electrodes at a given sampling rate, such as the sampling rate set for the multi-channel pulse oximeter 301(a), as described above.

In an embodiment, the sensor unit 201 may further comprise a tri-axis accelerometer 301(c). The tri-axis accelerometer may be used to track motion of a wearer. Every time the pulse oximeter 301(a) is sampled, so is the accelerometer 301(c) such that there are corresponding data points for every logged sample. The pulse oximeter 301(a) picks up signals caused by motion as well as signals derived from blood volume changes within vessels. The accelerometer 301(c) picks up signals caused by motion alone. Subtracting one from the other leaves signals due to blood volume changes. Within the device, signals from both the pulse oximeter 301(a) and accelerometer 301(c) are committed to on-device memory, are analysed on board the device, and are further process later via algorithms hosted on the online analysis platform 204.

In an embodiment, the sensor unit 301 may further comprise an internal temperature sensor 301(d). The internal temperature sensor 301(d) ensures that the sensing device 301 is running at a safe temperature. In addition to its standard use as a safety feature (whereby devices are powered down should they exceed a certain temperature threshold), the temperature data may be used to further refine system architecture over time by analysing trends in device temperature changes over time. Temperature data may be logged at regular intervals to infer additional information about patient activity.

In an embodiment, the sensor unit 301 may further comprise one or more circuits for noise reduction and/or signal amplification. The noise reduction circuit may comprise a low pass filter configured to remove motion induced low frequency noise on the sensor signal (e.g., signal from the pulse oximeter 301(a) and signal from the ECG sensor 301(b)). The signal amplification circuit may allow the sensor signal to be amplified to a certain level required by the processor unit 302.

In an embodiment, the processor unit 302 may comprise an arithmetic logic unit 302(a), a control unit 302(b), register arrays 302(c) and firmware 302(d). The arithmetic logic unit 302(a) performs arithmetic and logic operations guided by the control unit 302(b) on data from the input registers. The corresponding result is stored on an output register. The control unit 302(b) directs the operations of the process unit 302. It controls the logic behind arithmetic logic unit 302(a), the register arrays 302(c) and input and output devices on how to respond to the firmware instructions. Registers 302(c) are small amounts of storage within the processor unit 302. Input registers store data from the external sensors, e.g., sensors in the sensor unit 301, the control unit 302(b) guides the arithmetic logic unit 302(a) on what operations to perform on the data based on the firmware instructions and the corresponding results from the arithmetic logic unit 302(a) are stored in output registers for transfer back to the external sensors. The system firmware 302(d) controls the functionality of each of e.g., the inputs, storage, and power management on-board the device.

In an embodiment, the sensing device 201 may comprise at least two processor units, e.g., a primary microcontroller unit MCU and a secondary MCU. The two MCU units may be configured to work in a complementary manner.

In some cases, the primary MCU may be configured to continuously process and analyse samples from the pulse oximeter 301(a) and accelerometer sensor 301(c) which are then written to memory. The primary MCU may be configured to dynamically change the polling rate of these sensors based on off-nominal changes in readings i.e. those caused by motion artefacts. This ability assists in recording high quality heart waveforms, and obtaining accurate beat-to-beat timings such that on-board components or modules (e.g., cellular module 305(a), ECG sensor 301(b)) can be activated at an appropriate time. The primary MCU may be configured to dynamically alternate between sleep and active mode between sample collections. This greatly reduces energy consumption due to the low power requirements of the primary MCU while in an inactive state. Due to a multitude of superfluous features, the processors of comparable devices are required to process multiple tasks simultaneously, meaning the processor needs to be in active mode continuously, greatly reducing the overall runtime capabilities. Based on sensor readings, the primary MCU can trigger the secondary MCU to execute certain actions, such as, for example, entering sleep-mode when the device is not being worn.

The secondary MCU may be configured to process and analyse multiple sensor inputs (e.g., inputs from the pulse oximeter 301(a) and ECG sensor 301(b)), and conduct actions in response. The secondary MCU may remain in a low power sleep state until it is woken at predefined time intervals (to check if the device is being worn), or when triggered by the primary MCU. The primary MCU may be configured to execute ‘interrupts’ on the secondary MCU (e.g., instructing it to perform a different task based on sensor inputs). When the secondary MCU is woken from the sleep state after predefined time periods, it may for example take battery level readings, circuit performance readings, and ambient temperature readings, all of which are written to the devices memory. This data will be used internally to better understand how the device performs in various environments.

In an embodiment, the memory unit 303 may comprise a real-time clock 303(a), a random access memory 303(b) and an on-board memory 303(c). The real-time clock 303(a) keeps track of the time. The processor unit 302 may read this time and append a timestamp to every data log committed to the device's internal memory. The real-time clock 303(a) may be refreshed every time the sensing device 201 is returned to a charging station in order to compensate for time-slips (RTC's are highly accurate, but not perfect and need to be refreshed periodically). The random access memory 303(b) may be used to temporarily store the sampled data before it being arranged appropriately, encrypted by the processor unit 302, and eventually committed to long-term on-board memory 303(c). The on-board memory 303(c) may be in the form of flash storage designed to hold the gathered, encrypted data. This memory 303(c) may be wiped after successful confirmation of data uploading to the online portal. The write-rate of the on-board memory may be tailored to match the unusually high throughput demands caused by multiple streams of high-fidelity data.

In an embodiment, the power unit 304 may comprise a power management integrated circuit 304(a), a battery charger 304(b), and a battery 304(c). The power management integrated circuit 304(a) disseminates power amongst the various electronic components. The processor unit 302, receiving information from the sensor unit 301, can influence the functionality of the power management integrated circuit 304(a) as the sampling rates are controlled by applying varying power levels to the sensors of the sensor unit 301, which in turn impacts battery longevity. The battery charger may be capable of receiving power from the charging device 202 and enabling fast-charging of the device's battery 304(c). The battery 304(c) itself may possess a large charge capacity in order to satisfy the need of long term continuous heart rhythm monitoring. The large charge capacity (hence a large battery size) is enabled by the large internal volume of the sensing device 201 after many superfluous features that are typically adopted in existing wearable heart rate monitors are removed. In an embodiment, the battery 304(c) may have a charge capacity in the range between 1000 milliamp-hour (mAh) and 3000 mAh. In other embodiments, the charge capacity may be in the range between 1200 mAh to 2200 mAh.

In an embodiment, the power unit 304 may further comprise a low dropout regulator which may act to maintain a constant voltage to the processor unit 302 and the sensor unit 201 regardless of the level of charge of the battery (for example, at full charge, the battery may be running at 4.2 V, and when near fully discharged the battery may be running at 2.7 V. The low dropout regulator maintains a constant 2.5 V regardless of this); a DC-DC converter may work in parallel to the low dropout regulator to convert a source of one DC voltage level to another level; and a smart reset which may only allow current to flow once the voltage reaches e.g., 2.5 V from the battery. When this has been reached, the PMIC is reset and the low dropout regulator maintains a constant 2.5V.

In an embodiment, the interface unit 305 may comprise components or modules (not shown in FIG. 3) that enable connections between the sensing device 201 and the outside world for the purposes of e.g., human interaction, data transfer and power charging. In an embodiment, the interface unit 305 may comprise a cellular module 305(a) (e.g., a 3G module or a 4G/LTE module) configured to allow data exchange (e.g., uploading data) between the sensing device 201 and a remote server via an existing mobile network. In an embodiment, the remote server may be the same server on which the online analysis platform 204 is run. In an embodiment, the cellular module 305(a) may only be activated in a short period of time sufficient to allow at least part of the recorded data (e.g., ECG data) comprising one or more flagged anomalous events to be transmitted to the remote server. Selectively transmitting data that is indicative of heart rhythm anomalies in the middle of a long (e.g., 90 days) and continuous recording session allows such data to be seamlessly and hastily sent to the server and then e.g., a medical doctor should an anomaly meriting near-immediate intervention occur at any time.

The sensing device 201 may be worn on different parts of the body. In an embodiment, the sensing device 201 may be placed on the wrist (e.g., left wrist or right wrist) and may be in the form of a wrist band or strap. In an embodiment, the sensing device 201 may be placed on the arm (e.g., upper left arm or upper right arm) and may be in the form of an arm band or strap. The arm band or strap may be configured to accommodate one or more pairs of dry electrodes. The one or more pairs of dry electrodes may for example line the interior of the armband which will be positioned e.g., on the upper left arm. FIGS. 4A to 4C schematically depict an armband in accordance with an embodiment, which will be described below.

FIG. 4A is a plan view of the inner side of a detachable armband 400 in an unfolded state. As shown in the figure, the armband 400 may comprise two pulse oximeters 301(a)-1, 301(a)-2 and one ECG sensor 301(b) with eight pairs of dry electrodes 410a/410b, 420a/420b, 430a/430b, 440a/440b, 450a/450b, 460a/460b, 470a/470b, 480a/480b, all of which may be attached to the inner surface 491 of the armband 400. The light-emitting side of the pulse oximeters 301(a) may be in direct contact with the body skin. Note that other embodiments may comprise different numbers of the sensing devices and dry electrodes other than those shown in the figure. In an embodiment, the dry electrodes 410a/410b, 420a/420b, 430a/430b, 440a/440b, 450a/450b, 460a/460b, 470a/470b, 480a/480b may be in the form of concentric rings and may be made of a flexible polymer. Other different dry electrode designs that are commonly used for ECG are equally applicable.

FIG. 4B is a plan view of the outer side of the detachable armband 400 in the unfolded state. The armband 400 may comprise a plurality of batteries (e.g., six batteries 304(c)-1-304(c)-6 in this particular embodiment) and a main electrical circuit board (PCB) 493 comprising the ECG circuit of the ECG sensor 301(b). In the embodiment shown in FIGS. 4A and 4B, the main PCB 493, batteries 304(c)-1-304(c)-6 (abbreviated as 304(c) below), pulse oximeters 301(a)-1, 301(a)-2, and dry electrodes 410a/410b, 420a/420b, 430a/430b, 440a/440b, 450a/450b, 460a/460b, 470a/470b, 480a/480b may form the sensing device 201. In an embodiment, the pulse oximeters 301(a) may be mounted on the PCB 493 from the inner side of the armband 400. As shown in FIG. 4B, the plurality of batteries may be equally spaced along the length of the armband 400. In an embodiment, the batteries 304(c)-1, 304(c)-2, 304(c)-3, 304(c)-4, 304(c)-5, 304(c)-6 may be embedded in the body of the armband 400, the body of the armband 400 being defined as the part between the inner surface 491 and the outer surface 492.

Alternatively, in an embodiment, the plurality of batteries 494a-494f may be attached to the outer surface 492 of the armband 400. The pulse oximeters 301(a) and all the dry electrodes 410a/410b, 420a/420b, 430a/430b, 440a/440b, 450a/450b, 460a/460b, 470a/470b, 480a/480b may be electrically connected with the main PCB 493 which may be embedded in the body of the armband 400.

FIG. 4C is a side view of the detachable armband 400 in a folded state. As shown in the figure, the dry electrodes 410a/410b, 420a/420b, 430a/430b, 440a/440b, 450a/450b, 460a/460b, 470a/470b, 480a/480b may be carefully positioned on the armband 1000 such that when the armband is folded to tightly grip the left arm bicep, the two electrodes of each electrode pair are on the opposite sides of the arm and facing each other. Note that the electrodes 410a/450a, 420a/460a, 430a/470a, 440a/480a, 410b/450b, 420b/460b, 430b/470b, and 440b/480b are all spatially separated along the direction normal to the plane of the figure. As shown in the figure, the electrodes 410a, 420a, 430a, 440a, 450a, 460a, 470a, 480a and the respective corresponding electrodes 410b, 420b, 430b, 440b, 450b, 460b, 470b, 480b are located on the opposite side of the arm (not shown) and facing each other. Such an arrangement may maximise the distance between each electrode pair and thus maximise the electrical potential difference measured through the pair.

With reference to FIG. 5, the charging device 202 may possess data transfer and charging connection points complementary to the sensing device. The charging device 202 may comprise an AC-DC Converter 501 which converts the mains AC supply to DC power usable by the charging device 202, and a data transfer and charging circuitry unit 502 which is complementary to the contact points of the sensing device 201. The charging device 202 may be configured to comprise internally rerouted data transfer and charging connectors to receive and make contact with the metal contact points on the sensing device 201. When the two devices are in physical contact with each other, each of the metal contact points on the sensing device 201 connects to the corresponding metal contact point on the charging device 202 and thus a connection is established. A number of the metal contact points may be used for data transfer between the on-board memory 303(c) and the computing device 203. The other metal contact points may be used for charging. The complementary data transfer and charging connections between the sensing device 201 and the charging device 202 may be based on protocols such as for example USB 3.0, USB-C, or RS232.

In order to obtain high fidelity waveforms that are capable of revealing certain fine features in relation to heart rhythm abnormalities, such as for example the absence of the p-wave 110 and to enable the accurate triggering of the on-board ECG, the multi-channel pulse oximeter 301(a) and accelerometer 301(c) may preferably have a sampling rate between 215 Hz and 1000 Hz. In an embodiment, the sampling rate may be dynamically adjusted in the range between 215 Hz and 1000 Hz by the processor unit 302 (as controlled by the system firmware 302(d)) in response to a predetermined event. The predetermined event may be for example an increased physical activity, a change in signal quality, or a decreased battery level. For example, data measured by the accelerometer 301(c) may be used to at least partially determine the sampling rate through detecting increased activity levels, which the processor unit 302 responds to by increasing the sampling rate of both the accelerometer and pulse oximeter. In such a manner, small features which are easily disguised by motion-induced interference can be recovered more readily by processing algorithms used later in the online analysis platform 204. Moreover, when the quality of one or more sensor signals vary, the sampling rate may be also dynamically adjusted by the processor unit 302. For example, when the quality of the pulse oximeter signal degrades, the processor unit 302 may increase the sampling rate of both the accelerometer and pulse oximeter in order to substantially maintain a good data fidelity.

In parallel, a power-saving strategy may be employed which allows the sensing device 201 to decrease the rate of active sampling (e.g., from an initial rate of 1000 Hz to a later rate of 500 Hz) as battery-life decreases. The sampling rate may be reduced in a step-by-step manner and may be adjusted periodically. In an embodiment, the processor unit 302 may periodically check the remaining charge level of the battery 304(c) and calculate a percentage drop in the charge level over the last period of time. Then, the processor unit 302 may adjust/reduce the sampling rate by an amount proportional to the percentage change of the charge level of the battery 304(c). Fine control of the dynamic sampling rate contributes significantly to both the longevity of the battery and the fidelity of the data. With the help of the dynamic sampling control and a large capacity battery, the sensing device 201 is able to provide a single continuous recording period of up to 90 days, or approximately 90 days, or greater than 90 days, thereby significantly increasing the detection rate of heart rhythm abnormalities, such as AFib.

In an embodiment, the processor unit 302, or more specifically the firmware 302(d), may be configured to maintain a virtually constant sampling rate when the sampling rate decreases as a result of the decrease of the battery charge or the increase of the wearer's physical activity. The virtually constant sampling rate may be maintained by applying interpolation to the data obtained with a decreasing sampling rate. Interpolation offers the ability to reduce the live sampling rate gradually over time, as battery life decreases, whilst preserving accuracy of the waveform. The extent to which interpolation is used will vary depending on battery performance. It may be used from the beginning to end of recording duration if necessary, as battery degradation occurs over time with repeat uses of the same device. For example, when the sampling rate has reduced from e.g., initial 1000 Hz to current 500 Hz as a result of discharging of the battery 304(c), the number of measured data points per second dropped correspondingly from 1000 to 500.

As mentioned above, insufficient sampling results in poor temporal resolution and therefore low fidelity heart rhythm waveform and inaccurate ECG-triggering. Hence, to compensate for such data point loss, interpolation density is increased accordingly such that the total number of data points (measured and interpolated) per second is maintained to be 1000, corresponding to a constant sampling rate of 1000 Hz. Interpolation may be performed on the measured data by the sensing device 201 while the data is being recorded. Alternatively, it may be performed on data post-collection in order to reduce signal noise attributable to physical motion and other environmental factors. This processing may be performed on the data analysis platform 204 and may be supplemental to the electrical filtering performed on-board the device. In an embodiment, different interpolation methods may be applied to different parts of the measured data. For example, in the case where the measured sensor signal changes linearly with time, linear interpolation will be applied. In the case where the measured sensor signal changes nonlinearly with time, polynomial interpolation will be selected. The application of interpolation allows the sensor unit (e.g., the multi-channel pulse oximeter in this case) to be operated at low sampling rates (e.g., between 5 Hz and 215 Hz) while simultaneously maintaining a sufficient number of data points and therefore ensuring a high fidelity heart rhythm waveform and accurate ECG triggering.

In an embodiment, the firmware 302(d) may format the data appropriately, perform encryption, and log the encrypted data to the on-board memory 303(c) live. In an embodiment, data formatting may comprise further software-based filtering and smoothing (e.g., Savitzky-Golay smoothing). Data encryption may be obtained by means of e.g., a cryptographic algorithm (algebraic matrix-based).

In an embodiment, the sensing device 201 may immediately begin detecting a heartbeat signal when placed on a part of a wearer's body (e.g., upper left arm). As soon as the heartbeat signal is detected and simultaneously meets one or more predefined conditions, the sensing device 201 may automatically begin recording of the data which may have been filtered, formatted and encrypted by the hardware as well as firmware of the sensing device 201. The firmware 302(d) may incorporate an algorithm, which, when executed by the processor unit 302, may perform both self-starting and auto-reporting functions. The self-starting function may allow the sensing device 201 to determine whether the sensing device 201 can automatically start data recording while the self-reporting function may allow the sensing device 201 to transfer data captured during anomalous events. It may allow the captured data to be transferred immediately after the event, or as a batch on a daily basis. With reference to FIG. 4 and FIG. 6, the self-starting and auto-reporting algorithm 600 may comprise for example the following six steps.

At step 610, the processor unit 302 may determine whether the armband 400 is being worn. Both the pulse oximeters 301(a)-1, 301(a)-2 and the ECG sensor 301(b) may be activated. The processor unit 302 may command the ECG sensor 301(b) to periodically measure voltage differential across each electrode pair 410a/410b, 420a/420b, 430a/430b, 440a/440b, 450a/450b, 460a/460b, 470a/470b, 480a/480b and compare the measured voltage differential to a threshold level. In cases where one or more electrical voltage signals received from the ECG sensor 301(b) are higher than the threshold level, the processor unit 302 may determine that the armband 400 is being worn and may move to step 620. Otherwise, the processor unit 302 may stay at step 610.

At step 620, once the processor unit 302 determines the armband 400 is being worn, the processor unit 302 may initiate a full data recording session which may last for example 90 days. At this stage, only the pulse oximeters 301(a)-1, 301(a)-2 are activated while the ECG sensor 301(b) is set to the idling mode. The processor unit 302 may monitor peak-to-peak (P-P) pulse timings live and compute their regularity by comparing the P-P interval of the last detected beat with previous beats (e.g., the previous 5-10 beats).

At step 630, the processor unit 302 may determine whether any irregularity exists in the P-P pulse timings. This may be achieved for example by checking if the standard deviation of the P-P intervals rises over time. A rising standard deviation indicates either irregularity, or exercise-induced increase in heart rate. The latter can be ruled out via accelerometer measurements, for example. The processor unit 302 may determine the existence of irregularity for example by comparing the standard deviation to an ECG triggering threshold. If the standard deviation of the P-P intervals is lower than the ECG triggering threshold, the processor unit 302 may stay at step 630 and continue to monitor the P-P pulse timings. If the standard deviation of the P-P intervals is equal to or higher than the ECG triggering threshold, the processor unit 302 may proceed to step 640.

At step 640, the processor unit 302 may activate the ECG sensor 301(b) and initiate recording of ECG data. While the ECG data is being recorded, the pulse data (or PPG data) generated by the pulse oximeters 301(a)-1, 301(a)-2 is also being recorded and analysed in an unaffected manner. In an embodiment, the ECG data may be stored in the same on-board memory 303(c) where the pulse data (or PPG data) measured by the pulse oximeters 301(a)-1, 301(a)-2 is stored. In an embodiment, the ECG data may be stored in a different on-board memory 303(c).

The parallel operation of the pulse oximeters 301(a)-1, 301(a)-2 and the ECG sensor 301(b) may allow the ECG sensor 301(b) to be activated only when pulse irregularity is determined by the pulse oximeters 301(a)-1, 301(a)-2, thereby reducing the energy consumption of the sensing device 201. In an embodiment, the ECG sensor 301(b) may be deactivated by the processor unit 302 after a fixed duration of time, which may be, for example, a fixed duration of 10 minutes, a fixed duration of 20 minutes, a fixed duration of 30 minutes, or a fixed duration of 40 minutes. Alternatively, in an embodiment, the ECG sensor 301(b) may be deactivated if no irregularity in the P-P pulse timings had been identified in a most recent duration of time, which may be, e.g., a most recent 5 minutes, or a most recent 10 minutes. Once the ECG sensor 301(b) is deactivated, the processor unit 302 may stop the ECG data recording and move to step 650. Each individual ECG recording session may result in one or more ECG traces, each associated with one electrode pair.

At step 650, the processor unit 302 may activate the cellular module 305(a) so as to establish a connection between the remote server and the sensing device 201. Once the connection is established, the processor unit 302 may initiate transmission of the ECG data to the remote server via the cellular module 305(a). Along with the ECG data, other information associated with the sensing device 201, such as, for example, the remaining space in the memory unit, a diagnostic log of all the device components, and present battery level, may also be transmitted to the remote server. If the cellular module 305(a) cannot establish a connection with a remote server, an interrupt may be sent to the processor unit 302 to deactivate the cellular module 305(a) and maintain the current data in the on-board memory 303(c). Following a failed connection attempt, a number of additional attempts may be made periodically. Failing these, the processor unit 302 may continue to make attempts on a daily basis, for example, on the next daily scheduled secondary MCU transmission wake up. Upon completing the data transmission, the processor unit 302 may progress to step 660.

At step 660, the processor unit 302 may check if the cellular module 305(a) receives a signal from the remote server confirming the successful transmission of all data and may subsequently deactivate the cellular module 305(a). In an embodiment, the processor unit 302 may clear all the recorded ECG data in the on-board memory 303(c) so as to prepare for the next session of ECG recording.

After successfully receiving the ECG data, the remote server may perform further data processing and deterministic analysis on the processed ECG data to determine e.g., presence of AFib. The further data processing and deterministic analysis may be performed by an application run on the remote server, such as for example, the online analysis platform 204. The deterministic analysis is described in detail below with reference to FIG. 9.

Referring back to the self-starting and auto-reporting algorithm 600 shown in FIG. 6, at step 640, the ECG data may be generated in various different ways. In an embodiment, the ECG sensor 301(b) may be configured to generate multiple electrical voltage signals at any given point of time, each being obtained by measuring the electrical voltage between one pair of dry electrodes. Additionally or optionally, the ECG sensor 301(b) may be configured to associate each time-dependent electrical voltage signal to a corresponding pair of dry electrodes through which the electrical voltage signal is generated. Additionally or optionally, the ECG sensor 301(b) may be configured to perform real-time processing of the multiple electrical voltage signals. The real-time processing may comprise for example amplifying the originally weak electrical voltage signals and subsequently filtering the amplified electrical voltage signals. Note that, part or all of such real-time data processing may be performed by other on-board circuits, such as the aforementioned one or more circuits for noise reduction and/or signal amplification. All of the ECG traces may be recorded and stored in the on-board memory 303(c). The processor unit 302 may be configured to analyse the quality (e.g., signal to noise ratio) of each of the ECG traces and subsequently select the one with the best signal quality (e.g., signal to noise ratio). In comparison with the single electrode pair configuration, the advantage of having multiple electrode pairs is that more electrodes help improve the chances that one will record a good quality (e.g., high signal to noise ratio) ECG signal. However, simultaneous recording of multiple data streams coming from the multiple electrode pairs causes a high power consumption and consequently a faster battery drainage.

With reference to FIG. 7, in a preferred embodiment, the firmware 302(d) may incorporate a dynamic-switching algorithm 700 which is executed by the processor unit 302 at step 640, i.e. after an irregularity is detected in the P-P pulse timings by the sensing device 201 (e.g., the armband 400 shown in FIG. 4). The dynamic-switching algorithm 700 is able to reduce the power consumption of the ECG sensor 301(b) having multiple electrode pairs while simultaneously maintaining a good ECG data quality. The dynamic-switching algorithm 700 may comprise for example the following nine main steps.

Step 710: The processor unit 302 may activate the ECG sensor 301(b) by starting to periodically poll all of the electrode pairs (e.g., dry electrode pairs of the armband 400, 410a/410b, 420a/420b, 430a/430b, 440a/440b, 450a/450b, 460a/460b, 470a/470b, 480a/480b) of the ECG sensor 301(b) individually in quick succession to establish the noise level of each electrode pair. The polling rate may be for example between 100 Hz and 1000 Hz, between 100 Hz and 600 Hz, between 125 Hz and 550 Hz, or between 125 Hz and 512 Hz. The periodical polling of all of the electrode pairs may be maintained throughout an entire recording session.

Step 720: The processor unit 302 may establish the noise level of each electrode pair by calculating a signal-to-noise ratio (SNR) of the electrical voltage data collected through the corresponding electrode pair in a given time frame. The time frame for noise analysis may be for example between 0.1 second and 1 second.

The SNR of the electrical voltage data may be calculated using any conventional method SNR calculation. For example, obtaining an approximate estimate of SNR is achieved by first identifying a ‘clean’ beat cycle to use as a reference beat. This is done by evaluating the millivolt range of beat cycles until one is identified that contains low millivolt values between beat features. The noise is given by the root-mean-square error (RMSE) between each succeeding beat and the reference beat, while the signal is each beat's root-mean-square (RMS). The ratio is the product of the antecedent (signal value) divided by the consequent (noise value). This method is selected as it can also provide double function by supplying supporting evidence for an AFib onset, as AFib will skew RMSE and RMS values, which artificially increase noise values for the duration of the AFib run. This cannot be used to diagnose AFib as you cannot distinguish between arrhythmia.

Step 730: The processor unit 302 may compare the noise level of each electrode pair with a predefined noise threshold to identify if there is at least one electrode pair that has a noise level acceptable for data recording. In an embodiment, the predefined noise threshold may be a SNR threshold. The SNR threshold may have a value of at least 10:1, for example, 15:1, 20:1, 25:1 or 30:1. ECG data with a SNR of higher than 10:1 has the advantage of allowing p-waves to be distinguishable from noise when analysing each wave cycle individually.

If the electrical voltage data collected from an electrode pair in the given time frame has a SNR equal to or higher than the SNR threshold, the corresponding electrode pair will be regarded as an acceptable electrode pair. Whereas, if the electrical voltage data collected from an electrode pair in the given time frame has a SNR lower than the SNR threshold, the corresponding electrode pair will be regarded as an unacceptable electrode pair. When the processor unit 302 has identified at least one acceptable electrode pair, the processor unit 302 may progress to step 740. Otherwise, the processor unit 302 may progress to step 750.

Step 740: The processor unit 302 may select, among all the acceptable electrode pairs identified at step 730, the electrode pair having the lowest noise level (or the highest SNR in case of using a predefined SNR threshold, as described above) and may record the data collected from selected electrode pair. Once the ECG data recording has begun, the process unit 302 may progress to step 760.

Step 750: The processor unit 302 may turn on all of the electrode pairs (e.g., dry electrode pairs of the armband 400, 410a/410b, 420a/420b, 430a/430b, 440a/440b, 450a/450b, 460a/460b, 470a/470b, 480a/480b) for simultaneous recording, creating multiple simultaneous data streams depending on the number of electrode pairs in the sensing device 201 (e.g., eight data streams created for eight electrode pairs of the armband 400). In such a case, all the ECG data streams may be stored in the on-board memory 303(c) for transmission (see step 650 above). This scenario is avoided as a first-line approach due to the power-intensive nature of multi-ECG recording, which poses a risk to battery longevity if it is done frequently throughout the prescription period.

During the data recording, the SNR of each electrode pair remains continuously monitored by the processor unit 302 (steps 720 and 730). Should the SNR of one or multiple pairs return to being greater than the SNR threshold (e.g., 10:1), the electrode pair with the lowest noise will remain on while all other pairs will be switched off. This ensures battery longevity is sustained throughout the prescription period.

Step 760: Throughout the recording of the ECG data, the processor unit 302 may continue to periodically poll all of the electrode pairs at the above rate (e.g., between 125 Hz and 512 Hz) and perform noise analysis on the data collected in the given time frame so as to identify the electrode pair having the lowest noise level or the highest SNR. In the case where the electrode pair through which the ECG data is being recorded no longer has the highest SNR, the processor unit 302 may progress to step 770. Otherwise, the processor unit 302 may move back to step 740 and continue to record the data from the same electrode pair.

Step 770: The processor unit 302 may determine whether the electrode pair has been changed within a most recent period of time. The most recent period of time may be for example 3 seconds, 6 seconds or 9 seconds immediately before the time the processor unit 302 makes determination. In the case where there is no change in the electrode pair (i.e. no electrode pair switching) during the most recent e.g., 3 seconds, the processor unit 302 may progress to step 780. Otherwise, if the electrode pair has been changed at least once in the most recent e.g., 3 seconds, the processor unit 302 may progress to step 750.

Step 780: The processor unit 302 may be operable to end data recording for the electrode pair that has a degraded SNR and begin immediately recording data from the newly identified electrode pair having the highest SNR. The processor unit 302 may be operable to append the data collected from the newly identified electrode pair to the data collected from the previous pair such that a single ECG trace is constructed for transmission. The dynamic switching of electrode pairs during data recording according to their respective noise level ensures that only the data with the best quality (e.g., the lowest noise level or the highest SNR) is recorded. In such a way, the overall data quality is improved. This is in contrast with an ECG sensor having only one electrode pair where dynamic switching between electrode pairs is not possible.

Step 790: The processor unit 302 may check if the current recording session is concluded which may be determined by the expiry of a pre-defined time period of for example between 10 and 40 minutes, or when no irregularity in the P-P pulse timings had been identified in a most recent duration of time, which may be, e.g., a most recent 5 minutes, or a most recent 10 minutes.

At the end of each recording session, either a single ECG trace (e.g., step 780) or multiple ECG traces (e.g., step 750) will be generated and stored in the on-board memory 303(c). The dynamic-switching algorithm.

With reference to FIG. 2, in an embodiment, the computing device 203 may be a general purpose computer that is connected to the internet via an Ethernet cable. The computing device 203 may be connected to the charging device 202 via a USB cable to allow data transfer from the sensing device 201. In an embodiment, a dedicated software application may be installed on the computing device 203 which enables the communication between the computing device 203 and the online analysis platform 204. In an embodiment, the online analysis platform 204 may be accessed by a web browser installed on the computer device 203. In an embodiment, the data received from the sensing device 201 may be temporarily stored in a folder created for containing the data from this particular device. In an embodiment, the data stored in the sensing device 201 may be directly uploaded to the online analysis platform 204 via the dedicated software application or the web browser without being stored in the computing device. In this way, privacy protection of the system may be enhanced.

With continued reference to FIG. 2, the online analysis platform 204 may be a cloud based data hub which allows for development and implementation of various cloud-based algorithms. In an embodiment, the online analysis platform 204 may further comprise a data processing function block 801, a data analysis function block 802 and a database 803. The data processing function block 801 may comprise one or more data processing algorithms employed to decrypt the data, further process the data and generate a high fidelity heart rhythm waveform. The data analysis function block 802 may comprise one or more data analysis algorithms configured to perform deterministic analysis on the heart rhythm waveform and identify features that are capable of indicating any heart rhythm abnormalities. The data analysis function block 802 may also generate an analysis report based on findings of the analysis. The database 803 may be used to contain the high fidelity heart rhythm waveforms and the analysis report.

In an embodiment, upon receiving the data (i.e. the PPG data collected by the pulse oximeters 301(a) and the ECG data collected by the ECG sensor 301(b)), the data processing function block 801 may decrypt the data by means of a decryption key. The decryption key may be sensing device specific and thus can only be used to decrypt data from a particular sensing device. The decrypted data may be further processed by the data processing function block 801.

In an embodiment, in the case where the received ECG data comprises multiple ECG data streams (e.g., multiple data streams recorded at step 750), the data processing may comprise evaluating the SNR of each ECG data stream transmitted, and stitching together a single composite ECG trace from the cleanest snippets of traces from each electrode pair. Specifically, the data processing function block 801 may be operable to break each of the ECG data streams into a plurality of snippets (e.g., 30-second snippets). Since all the ECG data streams were recorded simultaneously by the sensing device 201, any snippet of one ECG data stream may have a corresponding snippet recorded in the same time frame in each of the other ECG data streams. In other words, for each recording time frame (e.g., 30-second time frame), there will be multiple snippets, each belonging to one ECG data stream. For each ECG data stream, the data processing function block 801 may be operable to calculate the noise level or the SNR of each of the plurality of snippets of the ECG data stream. Once all the ECG data streams have been processed in the same way, the data processing function block 801 may be operable to select the snippet having the lowest noise level or the highest SNR in any given time frame. Subsequently, the data processing function block 801 may concatenate all the selected snippets to generate a single composite ECG data stream. Such a stitched composite ECG data stream offers an improved overall data quality (e.g., a better overall SNR) over any of the original ECG data streams.

In an embodiment, the data processing function block 801 may leverage the individual ECG data streams along with the stitched composite stream to train a neural network to offer augmented processing of noisy ECG data streams. The ECG and PPG data streams will be broken into for example 30-second snippets for neural network processing to additionally establish the presence or absence of p-waves in these snippets. The PPG data can assist the neural network in discerning the position of peaks in the noisy ECG data streams.

The above-described processing method will not adversely affect the clinical usefulness of the produced ECG trace as it is constructed from raw data that was collected simultaneously from the same patient. Once generated, the single composite ECG trace may undergo further data processing and deterministic analysis as described below. The same applies to the case where the ECG data received by the online analysis platform 204 comprises a single ECG data stream (e.g., stitched data stream recorded at step 780).

In an embodiment, the data processing may further comprise data smoothing by means of e.g., Savitzky-Golay smoothing and/or moving average. The Savitzky-Golay smoothing is a low-pass filtering technique which attenuates higher frequency noise while suppressing low-frequency noise derived from wearer motion. This is a Finite Impulse Response (FIR) filter meaning its impulse response is of finite duration. In an embodiment, the data processing may comprise subtracting the data originated from the accelerometer 301(c) from the data originated from the multi-channel pulse oximeter 301(a) so as to remove or minimise the motion induced data interference. In an embodiment, the data processing function block 801 may perform recompiling of pulsatile coordinate data points so as to generate a corresponding full heart rhythm waveform, similar to the waveform shown in FIG. 1. The generated high fidelity heart rhythm waveform may then be stored in a directory created in the database 803 that is dedicated for storing the information of the wearer.

In an embodiment and with reference to FIG. 9, the deterministic analysis performed by the data analysis function block 802 may comprise three main steps: step 901, identifying features indicative of heart rhythm anomalies based on the generated pulse oximetry-derived heart rhythm waveform and ECG data (e.g., the data transmitted from the sensing device 201 to the remote server during the last continuous long-term recording); step 902, assigning evidence scores to the identified features; step 903, calculating an overall evidence score based on the previously assigned evidence scores; step 904, determining presence of heart arrhythmia based on the overall evidence score.

Specifically, when used to determine the presence of AFib, the deterministic analysis may be carried out in a way described below.

At step 901, the high fidelity heart rhythm waveform may be used for computation of systolic peak (R-wave 130), calculation of peak-to-peak intervals, interpolation of peak-to-peak times over a number of measured heart cycles to establish variance, and computational identification of the presence of features which may be indicative of a P-wave. The computation of systolic peak may be achieved via e.g., calculation of the positive-to-negative slope change on signals with an amplitude >75% of the maxima of all measured samples to that point (repeated for each waveform). The calculation of peak-to-peak intervals may be achieved via e.g., subtraction of the time measurements between the most recently identified R-wave 130 peak and the previously measured peak. The variance may be compared to an established acceptable range in order to identify anomalous variance. The computational identification of the presence of features may be achieved via e.g., negative-to-positive and positive-to-negative slope calculations on a set number of data points preceding identified systolic peaks;

At step 902, based on the calculation results obtained at step 901, flaggable anomalies of the heart rhythm waveform may be determined and an AFib evidence score may be assigned to each of them. The flaggable anomalies may comprise for example absence of features which may be indicative of a P-wave 110, and R-wave 130 variation outside of a predefined nominal range.

At step 903, the flagged data may be further processed for false-positive reduction. This may be achieved via e.g., analysis of flagged areas via derivative threshold algorithm analysed by a machine learning (ML) model such as support vector machine model (SVM). An SVM is a supervised learning model designed to be utilised with learning algorithms which examine data for classification and regression analysis, resulting in waveform features being detected with greater reliability. Analytical methods within this ML algorithm may comprise for example time-frequency examination, singular value decomposition, empirical mode decomposition, sparse signal recovery, and spectrum analysis for spectral-peak tracking. These methods may aid in obtaining usable data in the case that motion-induced interference is still present. The ML algorithm is essentially searching for sequences of R-R irregularity and patterns which indicate the presence or absence of P-waves 110, both of which are deduced as present or absent via the calculation of evidence scores. The strength of these evidence scores stems from data training sets that the ML algorithm has learned from. The post-processing flagged data may then be used for recalculation of AFib evidence scores and further training of the ML model.

At step 904, the final AFib evidence scores may be used to determine the presence of AFib and the relevant analysis data may be used for the compiling of a final report. The report may be for example in PDF format in which a red box may be drawn around the sectioned anomalous waveforms, and a timestamped notification, link, and explanation for the flagged anomaly may be shown.

In an embodiment, the system of FIG. 2 may be used to provide an end-to-end service for the diagnosis of AFib and other heart rhythm abnormalities. The end-to-end service may comprise for example the following three phases.

### Phase 1: Clinician-Initiated Device Provisioning

After a patient presents to a clinician with suspected heart rhythm abnormality, the clinician opens a browser-based referral portal (or the online analysis platform 204) and enters the patient's details. A patient directory is automatically created in the online portal upon clinician referral. The supplier or distributor of the sensing devices is notified of the referral, which is accepted by selecting the newly created directory and clicking ‘link device’. The online portal detects a technician removing a sensing 201 device up from a charging device 202, pairing the patient directory with that specific sensing device 201 automatically (in a similar fashion to how a computer detects the removal of a USB device). A decryption key is generated upon the directory association, used to decrypt data uploaded at the end of the collection period. The sensing device 201 is placed in a package along with other accessories, such as body attachment means (e.g. straps, bands (wristbands/armbands)) and user manual.

### Phase 2: Patient Monitoring

Upon receiving the sensing device 201, the patient attaches the sensing device 201 to their body. The sensing device 201 can be worn continuously for the duration of monitoring (up to 90 days). The sensing device 201 automatically begins data recording after a satisfactory heartbeat signal is detected. By the time a prescribed monitoring period has passed, the patient returns the sensing device 201 to the supplier.

### Phase 3: Data Collection and Analysis

Upon receiving the sensing device 202, the technician places the sensing device 201 into a charge device 202 and enables data uploading to the online portal. The uploaded data is decrypted with the unique decryption key and subsequently processed in a desired manner. An analysis report is generated after a deterministic analysis, e.g., the analysis process shown in FIG. 9, is performed on the processed data. The analysis report containing the information as to whether or not heart rhythm abnormalities are present is sent to the clinician for review.

In a different embodiment, any person may be able to buy a sensing device 201 along with a charging device 202 and other accessories (e.g., a wristband or an armband and a manual) from a local store or an online store. Upon receiving the sensing device, the person may register a user account on the online analysis platform 204 and create a personal profile by providing relevant personal information. Following the creation of the personal profile, the person may wear the sensing device and start recording his/her heart rhythm data. After a recommended recording period is over or the battery is discharged (e.g., 90 days), the sensing device 201 may be placed into the charging device 202 which is connected to a personal computer 203. The physical connection between the sensing device 201 and the charging device 202 may enable charging of the sensing device 201 as well as uploading the recorded data to the online analysis platform 204 either via a dedicated software application or a web browser. The online analysis platform 204 may perform deterministic analysis as shown in FIG. 9 and may provide the person with an evaluation result as to whether or not heart rhythm abnormalities (e.g., AFib) are detected during the last recording period. Relevant heart rhythm data may also be shown in order to support the finding/evaluation. The person may then send the evaluation result and the supporting data to a medical doctor (e.g., general practitioner (GP)) who will decide if a referral for specialist treatment is needed, for example.

1. A wearable device for detecting heart rhythm abnormalities, comprising:
at least one pulse oximeter configured to measure optically a bloodstream so as to monitor peak-to-peak (P-P) pulse timings; one ECG sensor having two or more pairs of dry electrodes and configured to measure two or more voltage signals in relation to heart rhythm activity, each voltage signal being measured across one electrode pair; and a processor unit;
wherein in response to an irregularity in P-P pulse timings, the processor unit is operable to: activate the ECG sensor by polling, periodically and sequentially, each of the two or more electrode pairs; determine an electrode pair for data recording; and record voltage signal data measured through the determined electrode pair.

2. A wearable device as claimed in claim 1, wherein to determine the electrode pair for data recording, the processor unit is operable to: calculate a signal-to-noise ratio (SNR) for each electrode pair based on voltage signal data measured through the corresponding electrode pair in a period of polling time; identify one or more electrode pairs that have a SNR equal to or higher than a SNR threshold; and select the electrode pair having the highest SNR from the identified one or more electrode pairs.

3. A wearable device as claimed in claim 2, wherein the processor unit is operable to repetitively perform calculating, identifying and selecting steps in parallel to the data recording.

4. A wearable device as claimed in claim 3, wherein the processor unit is operable to end data recording for the selected electrode pair when its SNR drops below the SNR threshold.

5. A wearable device as claimed in claim 4, wherein the processor unit is operable to select another electrode pair for data recording and determine that there is no change in the selected electrode pair during a predefined previous period of time.

6. A wearable device as claimed in claim 5, wherein the processor unit is operable to begin recording voltage signal data from the newly selected electrode pair.

7. A wearable device as claimed in claim 6, wherein the processor unit is operable to append the voltage signal data measured through the newly selected electrode pair to that measured through the previously selected electrode pair so as to form a single data stream.

8. A wearable device as claimed in claim 3, wherein the processor unit is operable to begin recording voltage signal data from all of the electrode pairs when none of the electrode pairs have a SNR equal to or higher than the SNR threshold, or when the processor unit determines that there is at least one change in the selected electrode pair during the predefined previous period of time.

9. A wearable device as claimed in claim 8, wherein the processor unit is operable to record and store multiple data streams each measured through one of the electrode pairs.

10. A wearable device as claimed in claim 8, wherein the processor unit is operable to end data recording for all of the electrode pairs when the process unit has identified and selected an electrode pair for data recording.

11. A wearable device as claimed in claim 10, wherein the processor unit is operable to select another electrode pair for data recording and determine there is no change in the selected electrode pair during the predefined previous period of time.

12. A wearable device as claimed in claim 11, wherein the processor unit is operable to begin recording voltage signal data from the newly selected electrode pair.

13. A wearable device as claimed in claim 1, wherein the wearable device is an armband; and wherein the two or more pairs of dry electrodes are attached to the inner surface of the armband and positioned such that when in use, the two dry electrodes of each electrode pair are on the opposite sides of the arm and facing each other.

14. A system for detecting heart rhythm abnormalities, comprising a wearable device as claimed in claim 1; an online analysis platform configured to process and analyse data transferred from the wearable device in order to determine heart rhythm abnormalities.

15. A system as claimed in claim 14, wherein when the data transferred from the wearable device comprises multiple data streams, the online analysis platform is operable to: break each of the data streams into a plurality of snippets, each snippet recorded in a different recording period; calculate a SNR for each of the plurality of snippets of each data stream; select the snippet having the highest SNR in every recording period; and concatenate all the selected snippets to generate a single composite data stream.

16. A method for improving ECG data quality, comprising: detecting an irregularity in P-P pulse timings by at least one pulse oximeters; activating an ECG sensor having two or more pairs of dry electrodes; polling, periodically and sequentially, each of the two or more electrode pairs; calculating a signal-to-noise ratio (SNR) for each electrode pair based on voltage signal data measured through the corresponding electrode pair in a period of polling time; identifying one or more electrode pairs that have a SNR equal to or higher than a SNR threshold; selecting the electrode pair having the highest SNR from the identified one or more electrode pairs; and recording voltage signal data measured through the determined electrode pair.

17. (canceled)

18. (canceled)
